(19) United States
(12) Patent Application Publication
(10) Pub. No.: US 2025/0107979 A1
(43) Pub. Date: Apr. 3, 2025

(54) Hair Treatment Compositions Having Anti-Dandruff Action

(71) Applicant: Henkel AG & Co. KGaA, Duesseldorf (DE)

(72) Inventors: Petra Westphal, Neu Wulmstorf (DE); Sabine Gruedl, Erkelenz (DE); Rainer Simmering, Grevenbroich (DE); Thomas Schroeder, Hamburg (DE); Dirk Hentrich, Hamburg (DE)

(21) Appl. No.: 18/906,741

(22) Filed: Oct. 4, 2024

Related U.S. Application Data
(63) Continuation of application No. PCT/EP2023/051054, filed on Jan. 18, 2023.

(30) Foreign Application Priority Data
Apr. 7, 2022 (DE) 102022203484.8

(51) Int. Cl. A61K 8/34; A61K 8/37; A61K 8/49; A61Q 5/00

(52) U.S. Cl. CPC A61K 8/345; A61K 8/37; A61K 8/375; A61K 8/4926; A61Q 5/006

(57) ABSTRACT

The invention relates to hair treatment agents based on a specific active ingredient combination of a) and b), in which a) represents compounds of the following formula (I) R¹—O—[CH₂—CH(OH)—CH₂—O]ₙ—H (I), where R¹ is a branched C₆-C₁₆ alkyl, alkenyl, or alkynyl group and n is a number from 1-3, and b) represents at least one active anti-dandruff ingredient which differs from a). The hair treatment compositions according to the invention are suitable for treating dandruff.

### FIELD OF THE INVENTION

The present invention relates to hair treatment agents based upon a special active ingredient combination for controlling dandruff and to the cosmetic use of the hair treatment agents for cleaning hair and for the prophylaxis, reduction, elimination, and alleviation of dandruff on hair-covered body surfaces. The present invention further relates to a cosmetic method for controlling scalp dandruff using the agents.

Even mild dandruff on the scalp or other hair-covered areas of the body is perceived as a sign of a lack of care and hygiene. The formation of dandruff is usually accompanied by painful itching, which can cause scratching reactions and lead to damage to the affected areas of skin. Skin damage in turn often forms the basis for infections and pathogens.

Dandruff can be promoted by various factors—for example, hereditary predisposition, a tendency towards increased sebum production (seborrhea), hormonal fluctuations, stress, climatic conditions, or improper hair care. However, the decisive contribution to dandruff formation is made by the colonization of the scalp with yeast fungi of the genus Malassezia.

### BACKGROUND OF THE INVENTION

In the fight against dandruff, efforts have therefore always been made to reduce and/or eliminate the bacterial colonization of the scalp with Malassezia.

For this purpose, numerous cosmetically acceptable and effective active antidandruff ingredients are known from the prior art, such as piroctone olamine, climbazole, zinc pyrithione or other zinc salts, sulfur and/or selenium sulfide.

The effect of these active antidandruff ingredients is mainly based on their fungicidal properties.

However, previously mentioned conventional active antidandruff ingredients have disadvantages. On the one hand, they can in some cases be irritating to the skin, and, due to their poor solubility in water, they are somewhat complicated to incorporate into conventional cosmetic products. In addition, due to their poor biodegradability, they are under increasing pressure and are subject to increasingly stringent regulations or banned from use in cosmetic products.

However, a reduction in the quantity or even a complete replacement of undesired or problematic substances in cosmetic products can only be carried out if alternatives are available that also have the desired properties for the intended use.

Various plant extracts, e.g., arnica, birch, burdock root, beard lichen, poplar, stinging nettle, myrtle, or hazelwort extracts, are known to be active anti-dandruff ingredients based upon natural substances. However, with regard to their immediate and/or long-lasting effectiveness, these extracts cannot completely replace the classic active antidandruff ingredients mentioned above.

There is therefore still a need for compounds having good efficacy against dandruff that can be obtained from natural sources and can be combined with classic active anti-dandruff ingredients to form potent or enhanced anti-dandruff agents.

### BRIEF SUMMARY OF THE INVENTION

The object of the invention is accordingly to provide a cosmetic hair treatment agent having anti-dandruff action, the performance of which is enhanced, and which comprises a very high proportion of environmentally friendly, active anti-dandruff ingredients.

In addition to improved anti-dandruff action, enhanced performance also means gentle cleaning and/or care of the scalp and hair.

The objects of the invention are achieved by the subject matter of claim 1.

The combination of at least two different active anti-dandruff ingredients a) and b) comprising at least one specific glycerol ether resulted in hair treatment agents
with increased, accelerated, and/or prolonged anti-dandruff action, which is understood to mean the effective treatment of dandruff conditions—in particular, those associated with a colonization and/or proliferation of yeast fungi of the genus Malassezia,
with a reduced potential for skin irritation,
with improved environmental compatibility,
with improved care properties for the treated hair.

### DETAILED DESCRIPTION OF THE INVENTION

The present invention is therefore characterized by the following statements:
1. A hair treatment agent, containing in a cosmetic carrier
a) at least one compound of the following formula (I)

R¹—O—[CH₂—CH(OH)—CH₂—O]ₙ—H (I)
where
   R¹ represents a branched C₆-C₁₆ alkyl, alkenyl, or alkynyl group
   n represents a number from 1-3, and
b) at least one active anti-dandruff ingredient different from a).
2. The hair treatment agent according to statement 1, containing the active ingredients a) and b) in a weight ratio ranging from 5:1 to 1:1.
3. The hair treatment agent according to one of the preceding statements, containing the active ingredients a) and b) in a weight ratio ranging from 4:1 to 2:1.
4. The hair treatment agent according to one of the preceding statements, wherein the total amount of the or each compound according to formula (I) ranges from 0.1-3 wt. % relative to the total weight of the hair treatment agent.

5. The hair treatment agent according to one of the preceding statements, wherein the total amount of the or each compound according to formula (I) ranges from 0.3-1.5 wt. % relative to the total weight of the hair treatment agent.
6. The hair treatment agent according to one of the preceding statements, wherein n is the number 1 in the or each compound according to formula (I).
7. The hair treatment agent according to one of the preceding statements, wherein, in the or each compound according to formula (I), R¹ represents a branched C₆-C₁₂ alkyl, alkenyl, or alkynyl group.
8. The anti-dandruff hair treatment agent according to one of the preceding statements, wherein, in the or each compound according to formula (I), R¹ represents a branched C₆-C₁₀ alkyl, alkenyl, or alkynyl group.
9. The anti-dandruff hair treatment agent according to one of the preceding statements, wherein, in the or each compound according to formula (I), R¹ represents a branched C₈ alkyl, alkenyl, or alkynyl group.
10. The hair treatment agent according to one of the preceding statements, wherein the compound according to formula (I) is H₃C—(CH₂)₅—CH(CH₃)—O—CH₂—CH(OH)—CH₂OH (methylheptylglycerin).
11. The hair treatment agent according to one of the preceding statements, wherein the total amount of the at least one active anti-dandruff ingredient b) different from a) ranges from 0.05-1 wt. % relative to the total weight of the hair treatment agent.
12. The hair treatment agent according to one of the preceding statements, wherein the total amount of the at least one active anti-dandruff ingredient b) different from a) ranges from 0.05-0.5 wt. % relative to the total weight of the hair treatment agent.
13. The hair treatment agent according to one of the preceding statements, wherein the or each active anti-dandruff ingredient b) is selected from piroctone olamine, zinc pyrithione, zinc oxide, zinc carbonate, climbazole, ketoconazole, salicylic acid, sulfur, selenium sulfide, tar preparations, undecenoic acid derivatives, lactic acid esters (Sodium Lauroyl Lactylate, Sodium Caproyl Lactylate, Sodium Caproyl/Lauroyl Lactylate), 3-hydroxypropyloctanoates (Propanediol Caprylate), burdock root extracts, poplar extracts, stinging nettle extracts, walnut shell extracts, birch extracts, willow bark extracts, beard lichen extracts, myrtle extracts, hazelwort extracts, rosemary extracts, arnica extracts, fruit extracts of black pepper (Piper nigrum Fruit Extract), bark extracts of mimosa plants (Inga Alba Bark Extract), extracts from cinnamon bark (Cinnamomum cassia Bark Extract), tangerine peel extracts (Citrus reticulata (Tangerine) Peel Extract), lemon flower oil, lemon leaf oil, lemon fruit oil (Citrus limon (Lemon) Flower/Leaf/Stem Oil), extracts from mint (Mentha arvensis Extract), or mixtures thereof.
14. The hair treatment agent according to one of the preceding statements, wherein the or each active anti-dandruff ingredient b) is selected from piroctone olamine, propanediol caprylate, sodium caproyl/lauroyl lactylate, or mixtures thereof.
15. The hair treatment agent according to one of the preceding statements, wherein the active anti-dandruff ingredient b) is piroctone olamine.
16. The hair treatment agent according to one of statements 1 to 14, wherein the active anti-dandruff ingredient b) is propanediol caprylate.
17. The hair treatment agent according to one of statements 1 to 14, wherein the active anti-dandruff ingredient b) is sodium caproyl/lauroyl lactylate.
18. The hair treatment agent according to one of the preceding statements, containing 0.5-20 wt. % of at least one surfactant c) selected from anionic surfactants, amphoteric surfactants, zwitterionic surfactants, non-ionic surfactants, cationic surfactants, or mixtures thereof.
19. The hair treatment agent according to one of the preceding statements, containing
c1) 1-15 wt. % of at least one anionic surfactant,
c2) 0-7.5 wt. % of at least one amphoteric or zwitterionic surfactant,
c3) 0-5 wt. % of at least one non-ionic surfactant, wherein the quantities specified relate to the total weight of the hair treatment agent.
20. The hair treatment agent according to one of the preceding statements, containing 0.01-3 wt. % of at least one cationic polymer d), wherein the quantities specified relate to the total weight of the hair treatment agent.
21. The hair treatment agent according to statement 20, wherein the total amount of the or each cationic polymer d) ranges from 0.02-2 wt. % relative to the total weight of the hair treatment agent.
22. The hair treatment agent according to one of statements 20 or 21, wherein the total amount of the or each cationic polymer d) ranges from 0.025-1 wt. % relative to the total weight of the hair treatment agent.
23. The hair treatment agent according to one of statements 20 to 22, wherein the or each cationic polymer d) is selected from compounds known under the INCl names, Guar Hydroxypropyltrimonium Chloride, Polyquaternium-10, Polyquaternium-6, Polyquaternium-67, Polyquaternium-37, or mixtures thereof.
24. The hair treatment agent according to one of the preceding statements, containing 0.01-5 wt. % of at least one hair care substance e) different from d), wherein the quantities specified weight to the total weight of the hair treatment agent.
25. The hair treatment agent according to statement 24, wherein the total amount of the or each hair care substance e) different from d) ranges from 0.025-3 wt. % relative to the total weight of the hair treatment agent.
26. The hair treatment agent according to one of statements 24 or 25, wherein the total amount of the or each hair care substance e) different from d) ranges from 0.05-2.5 wt. % relative to the total weight of the hair treatment agent.
27. The hair treatment agent according to one of statements 24 to 26, wherein the or each hair care substance e) different from d) is selected from glycerol esters, natural oils, butters or waxes, ester oils, ethers, C₈-C₁₆ alkanes, or mixtures thereof.
28 The hair treatment agent according to one of statements 24 to 27, wherein the hair care substance e) different from d) is glycerol monooleate.
29. A cosmetic use of a hair treatment agent according to one of statements 1 to 28 for cleaning and caring for human hair, and for the prophylaxis, reduction, elimination, and alleviation of dandruff on hair-covered body surfaces.
30. The cosmetic use according to statement 29, wherein the hair treatment agent is an antidandruff shampoo.
31. A cosmetic method for controlling scalp dandruff, in which a hair treatment agent according to one of statements 1 to 28 is applied to the hair and the scalp and
   rinsed out again either after a contact time of 5 seconds to 5 minutes, or
   left on the hair and scalp until the next hair washing.

As a first substantial constituent, the hair treatment agents according to the invention contain at least one compound according to formula (I).

Such biobased glycerol ethers are suitable as preservatives in cosmetic or pharmaceutical compositions and as odor control agents in deodorants (WO 2021/237207 A1).

The antidandruff action of medium-chain glycerol ethers according to formula (I) has hitherto not been known, nor has its activity as a booster for other active antidandruff ingredients.

The hair treatment agents according to the invention preferably contain compounds according to formula (I), in which n represents the number 1. Preference is also given to compounds according to formula (I), in which R¹ represents a branched C₆-C₁₂ alkyl, alkenyl, or alkynyl group, more preferably a branched C₆-C₁₀ alkyl, alkenyl, or alkynyl group, and particularly preferably a branched C₈ alkyl, alkenyl, or alkynyl group.

A particularly preferred compound according to formula (I) is H₃C—(CH₂)₅—CH(CH₃)—O—CH₂—CH(OH)—CH₂OH (methylheptylglycerin-MHG).

It has been found that even small amounts of compounds according to formula (I) are sufficient to achieve excellent antidandruff action in combination with at least one other active antidandruff ingredient b).

It is particularly advantageous that even small amounts of compounds according to formula (I) in the hair treatment agents according to the invention are sufficient to boost the action of further active antidandruff ingredients b), whereby their content can be significantly reduced.

In addition to the amounts of active antidandruff ingredients a) and b), their weight ratio also plays a role. An excess of compounds according to formula (I) (relative to the other active antidandruff ingredient b)) has been shown to be advantageous for achieving optimal antidandruff action.

A weight ratio of the active ingredients a) and b) therefore preferably ranges from 5:1 to 1:1, and particularly preferably from 4:1 to 2:1.

Likewise, a total amount of one or more compound(s) according to formula (I) of 0.1-3 wt. % relative to the total weight of the hair treatment agent is preferred. Particularly preferred are total amounts of one or more compound(s) according to formula (I) of 0.2-2 wt. %, and in particular 0.3-1.5 wt. %.

Very particular preference is given to using methylheptylglycerin (MHG) in the aforementioned amounts and weight ratios in the hair treatment agents according to the invention—in particular, biobased methylheptylglycerin, which can be obtained from sustainable, renewable, plant-based raw materials.

Corresponding products are commercially available—for example, from Inolex under the trade name, Lexgard® Natural MHG MB.

As at least one active antidandruff ingredient b), hair treatment agents according to the invention can preferably contain one or more active ingredients from the following groups:
b1) piroctone olamine,
b2) zinc pyrithione,
b3) zinc oxide,
b4) zinc carbonate,
b5) climbazole,
b6) ketoconazole,
b7) salicylic acid,
b8) sulfur,
b9) selenium sulfide,
b10) tar preparations,
b11) undecenoic acid derivatives,
b12) lactic acid esters (Sodium Lauroyl Lactylate, Sodium Caproyl Lactylate, Sodium Caproyl/Lauroyl Lactylate),
b13) 3-hydroxypropyloctanoates (Propanediol Caprylate),
b14) burdock root extracts,
b15) poplar extracts,
b16) stinging nettle extracts,
b17) walnut shell extracts,
b18) birch extracts,
b19) willow bark extracts,
b20) rosemary extracts,
b21) arnica extracts,
b22) fruit extracts of black pepper (Piper nigrum Fruit Extract),
b23) extracts from the bark of mimosa plants (Inga Alba Bark Extract),
b24) extracts from cinnamon bark (Cinnamomum cassia Bark Extract),
b25) tangerine peel extracts (Citrus reticulata (Tangerine) Peel Extract),
b26) lemon flower oil, lemon leaf oil, lemon fruit oils (Citrus limon (Lemon) Flower/Leaf/Stem Oil),
b27) extract from mint (Mentha arvensis Extract),
b28) or mixtures thereof.

A particularly advantageous antidandruff action in conjunction with excellent skin compatibility was achieved when compounds of the formula (I)—in particular, methylheptylglycerin-were combined with one or more active antidandruff ingredients b) from the groups b1), b12), b13), or mixtures thereof.

In the sense of the present invention, the combinations a) and b) are very particularly preferred:
methylheptylglycerin and piroctone olamine,
methylheptylglycerin and propanediol caprylate,
methylheptylglycerin and piroctone olamine and propanediol caprylate,
methylheptylglycerin and sodium caproyl/lauroyl lactylate,
methylheptylglycerin and piroctone olamine and sodium caproyl/lauroyl lactylate.

The at least one active antidandruff ingredient b) can be used in the hair treatment agents according to the invention in a total amount of 0.05 to 1 wt. %, more preferably 0.05 to 0.9 wt. %, particularly preferably 0.05 to 0.75 wt. %, very particularly preferably 0.05 to 0.6 wt. %, and in particular 0.05 to 0.5 wt. %, wherein the quantities specified relate to the total weight of the hair treatment agents.

Daily use of some commercial antidandruff compositions (for example those comprising only higher amounts of zinc pyrithione) can sometimes lead to dehydration and, in the worst case, increased dandruff-especially in the case of sensitive scalps.

The hair treatment agents according to the invention do not have this disadvantage. They can be used regularly and reduce, eliminate, alleviate, and/or prevent the formation of (scalp) dandruff. Even in the case of small amounts of active antidandruff ingredients a) and b) used, the effect occurs, for example, in hair treatment agents that, based upon their weight, comprise up to 1 wt. % of components a) and b) (with an excess of active ingredient a), as explained above).

The hair treatment agents according to the invention preferably contain active ingredients a) and b) in a cosmetic carrier. In the context of the invention, this is understood to be preferably an aqueous or aqueous-alcoholic carrier.

The cosmetic carrier preferably contains at least 70 wt. %, particularly preferably at least 72.5 wt. % and more particularly preferably at least 75 wt. %, water.

Furthermore, the cosmetic carrier may contain 0.01 to 10 wt. %, preferably 0.05 to 9 wt. %, and in particular 0.10 to 6 wt. %, of at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, phenoxyethanol, or mixtures of said alcohols.

The hair treatment agents according to the invention can in principle be packaged both as rinse-off hair cleaning agents and as rinse-off or leave-on hair conditioning agents and preferably contain, in addition to the active antidandruff ingredients a) and b), 0.5-20 wt. % of at least one surfactant that can be selected from anionic, amphoteric, zwitterionic, non-ionic, and cationic surfactants, or any mixtures thereof.

Preference is given to hair treatment agents according to the invention that are packaged as rinse-off hair cleaning agents—for example, as antidandruff shampoos.

Mild and good foaming properties of rinse-off hair cleaning agents according to the invention can be controlled by carefully selecting surfactant amounts and/or surfactant types.

Preferably, rinse-off hair cleaning agents according to the invention contain at least one anionic surfactant, which is responsible for producing satisfactory foam amounts and foaming properties. To achieve an optimal balance between mildness and foaming properties, a mixture of at least one anionic surfactant and at least one mild co-surfactant is particularly preferred. The mild co-surfactant can preferably be selected from amphoteric and/or zwitterionic and/or non-ionic surfactants.

In a preferred embodiment, hair treatment agents according to the invention are packaged as rinse-off hair cleaning agents and contain
c1) 1-15 wt. % of at least one anionic surfactant,
c2) 0-7.5 wt. % of at least one amphoteric or zwitterionic surfactant,
c3) 0-5 wt. % of at least one non-ionic surfactant,
wherein the quantities specified relate to the total weight of the hair treatment agent.

Suitable anionic surfactants within the meaning of the present invention are:
linear and branched fatty acids having 8 to 30 C atoms (soaps);
ether carboxylic acids of the formula R—O—(CH₂—CH₂O) x-CH₂—COOH, in which R is a linear or branched, saturated or unsaturated alkyl group having 8 to 30 C atoms, and x=0 or 1 to 16;
acyl sarcosides having 8 to 24 C atoms in the acyl group (sarcosinate surfactants);
acyl taurides having 8 to 24 C atoms in the acyl group (taurate surfactants);
acyl isethionates having 8 to 24 C atoms in the acyl group (isethionate surfactants);
sulfosuccinic acid mono- and/or dialkyl esters having 8 to 24 C atoms in the alkyl group and sulfosuccinic acid monoalkyl polyoxyethyl esters having 8 to 24 C atoms in the alkyl group and 1 to 6 oxyethyl groups (sulfosuccinate surfactants);
alpha-olefin sulfonates having 8 to 24 C atoms (alpha-olefin sulfonate surfactants);
alkyl sulfates and/or alkyl ether sulfate salts of the formula R—(OCH₂—CH₂)ₙ—O—SO₃X in which R preferably means a straight-chain or branched, saturated or unsaturated alkyl group having 8 to 30 C atoms, x is the number 0 or 1 to 12, and X denotes an alkali, alkaline earth, ammonium, or alkanolamine ion;
sulfonates of unsaturated fatty acids having 8 to 24 C atoms and 1 to 6 double bonds,
esters of tartaric acid and citric acid with alcohols, which represent addition products of about 2-15 molecules ethylene oxide and/or propylene oxide to fatty alcohols having 8 to 22 C atoms, and/or
alkyl and/or alkenyl ether phosphates of the formula

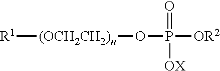

in which R¹ preferably represents an aliphatic hydrocarbon functional group having 8 to 30 carbon atoms, R² represents hydrogen, a functional group (CH₂CH₂O)ₙR¹ or X, n represents numbers from 0 to 10, and X represents hydrogen, an alkali or alkaline earth metal, or the group —NR³R⁴R⁵R⁶, where R³ to R⁶ represent, independently of one another, a C₁ to C₄ hydrocarbon functional group.

Particular preference is given to alkylsulfate and/or alkylether sulfate salts, (salts of) ether carboxylic acids, sarcosinates, isethionates, taurates, sulfosuccinates and/or alpha-olefin sulfonates, in particular alkylsulfate and/or alkylether sulfate salts.

Suitable amphoteric and/or zwitterionic surfactants within the meaning of the present invention are in particular mild surfactants having excellent foaming properties.

These are preferably understood to be surface-active compounds selected from the group comprising alkylbetaines, alkylamidoalkylbetaines, alkyl amphoacetates, alkyl amphodiacetates, alkyl amphopropionates, alkyl amphodipropionates, alkylsultaines, alkylhydroxysultaines, alkylamine oxides, alkyl amphoglycinates, alkyl iminodiacetates, alkyl iminodipropionates, alkyl amphopropyl sulfonates, alkyl amphocarboxyglycinates and alkyl amphocarboxypropionates.

Suitable alkylbetaines and/or alkylamidopropylbetaines preferably comprise C₄-C₂₄, more preferably C₆-C₁₈, particularly preferably C₈-C₁₄ alkyl chains which may be linear or branched, wherein linear is preferred. Particularly preferred alkylbetaines or alkylamidopropylbetaines are selected from the group of lauryl betaines, coco betaines, behenyl betaines, caprylic/capramidpropyl betaines, cetyl betaines, cocamidoethyl betaines, cocamidopropyl betaines, coco/oleamidopropyl betaines, decyl betaines, dimer dilinoleamidopropyl dibetaines, hydrogenated tallow betaines, hydroxylauryl/hydroxymyristyl betaines, isostearamidopropyl betaines, lauramidopropyl betaines, lauryl betaines, myristyl betaines, oleamidopropyl betaines, oleyl betaines, palmitamidopropyl betaines, ricinoleamidopropyl betaines, stearamidopropyl betaines, stearyl betaines, tallowamidopropyl betaines, tallow betaines, tallow dihydroxyethyl betaines, undecylenamidopropyl betaines, sunflower amidopropyl betaines, cetyl betaines, lauryl betaines.

Very particular preference is given to cocamidopropyl betaine.

Particularly suitable alkyl amphoacetates, alkyl amphodiacetates, alkyl amphopropionates or alkyl amphodipropionates are selected from the group of cocobetainamido amphopropionate, DEA-cocoamphodipropionate, disodium caproamphodiacetate, disodium caproamphodipropionate, disodium capryloamphodiacetate, disodium capryloamphodipropionate, disodium cocoamphodiacetate, disodium cocoamphodipropionate, disodium isostearoamphodiacetate, disodium isostearoamphodipropionate, disodium laureth-5 carboxyamphodiacetate, disodium lauroamphodiacetate, disodium lauroamphodipropionate, disodium oleoamphodipropionate, disodium PPG-2-isodeceth-7 carboxyamphodiacetate, disodium stearoamphodiacetate, disodium tallowamphodiacetate, sodium caproamphopropionate, sodium capryloamphoacetate, sodium capryloamphopropionate, sodium cocoamphoacetate, sodium cocoamphopropionate, sodium isostearoamphoacetate, sodium isostearoamphopropionate, sodium lauroamphoacetate, sodium lauroamphopropionate, sodium myristoamphoacetate, sodium oleoamphoacetate, sodium oleoamphopropionate, sodium stearoamphoacetate, sodium stearoamphopropionate, sodium tallamphopropionate, sodium tallowamphoacetate, sodium undecylenoamphoacetate and sodium undecylenoamphopropionate.

Very particular preference is given to sodium cocoamphoacetate and disodium cocoamphodiacetate.

Particularly suitable alkylsultaines or alkylhydroxysultaines are selected from the group of capryl sultaine, cocamidopropyl hydroxysultaine, coco-hydroxysultaine, coco-sultaine, erucamidopropyl hydroxysultaine, lauramidopropyl hydroxysultaine, lauryl hydroxysultaine, lauryl sultaine, myristamidopropyl hydroxysultaine, oleamidopropyl hydroxysultaine, tallowamidopropyl hydroxysultaine.

Particularly suitable alkylamine oxides are selected from the group of behenamine oxide, cocamidopropylamine oxide, cocamine oxide, decylamine oxide, decyltetradecylamine oxide, dihydroxyethyl C8-10 alkoxypropylamine oxide, dihydroxyethyl C9-11 alkoxypropylamine oxide, dihydroxyethyl C12-15 alkoxypropylamine oxide, dihydroxyethyl cocamine oxide, dihydroxyethyl lauramine oxide, dihydroxyethyl stearamine oxide, dihydroxyethyl tallowamine oxide, hydrogenated tallowamine oxide, isostearamidopropylamine oxide, lauramidopropylamine oxide, lauramine oxide, myristamidopropylamine oxide, myristamine oxide, palmitamidopropylamine oxide, palmitamine oxide, PEG-3 lauramine oxide, stearamidopropylamine oxide, stearamine oxide, tallowamidopropylamine oxide, tallowamine oxide, undecylenamidopropylamine oxide.

Particularly suitable alkyl amphoglycinates are selected from the group of caproamphoglycinate, capryloamphoglycinate, cocoamphoglycinate, myristoamphoglycinate, tallowamphoglycinate, isostearoamphoglycinate, lauroamphoglycinate, oleoamphoglycinate, stearoamphoglycinate, undecylenoamphoglycinate.

Particularly suitable alkyliminodiacetates or alkyliminodipropionates are selected from the group of disodium cocaminopropyl iminodiacetate, disodium hydroxyethyliminodiacetate, disodium lauriminodiacetate, disodium lauriminodipropionate, disodium steariminodipropionate, disodium tallowiminodipropionate, sodium C12-15 alkoxypropyl iminodipropionate, sodium cocoiminodiacetate, sodium lauriminodipropionate.

Particularly suitable alkyl amphopropyl sulfonates are selected from sodium cocoamphohydroxypropylsulfonate, sodium lauroamphohydroxypropylsulfonate, sodium oleoamphohydroxypropylsulfonate, sodium stearoamphohydroxypropylsulfonate, disodium lauriminobishydroxypropylsulfonate, disodium cocoamphocarboxyethylhydroxypropylsulfonate, sodium caproamphohydroxypropylsulfonate, sodium capryloamphohydroxypropylsulfonate.

Hair treatment agents according to the invention that are packaged as hair cleaning agents preferably contain alkylamidoalkylbetaines, alkyl amphoacetates and/or alkyl amphodiacetates as amphoteric and/or zwitterionic surfactants.

Suitable non-ionic surfactants for use as a co-surfactant are, for example,
amine oxides, which can be selected from compounds of the general formulae (I) or (II)

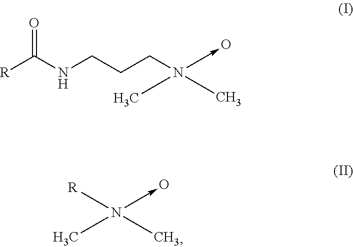

in which R in each case represents a straight-chain or branched, saturated or monounsaturated or polyunsaturated alkyl or alkenyl group having 6 to 24 carbon atoms, preferably having 8 to 18 carbon atoms.

The surfactants of the aforementioned formulae (I) or (II) that are known under the INCl names Cocamine Oxide, Lauramine Oxide, and/or Cocamidopropylamine Oxide and are commercially available from a number of suppliers are preferred in particular.
fatty acid alkanolamides of the following general formula,

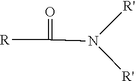

in which R preferably signifies a linear or branched saturated or unsaturated alkyl or alkenyl residue having 8 to 24 carbon atoms, and the residues R′ denote hydrogen or the group-(CH₂)ₙOH, in which n signifies the number 2 or 3, with the proviso that at least one of the residues R′ denotes the aforementioned residue-(CH₂)ₙOH,
mixtures of alkyl (oligo) glucosides and fatty alcohols, for example, the commercially available product Montanov®68,
sterols. Sterols are understood to refer to a group of steroids that bear a hydroxy group at the C atom 3 of the steroid structure, and are isolated both from animal tissue (zoosterols) and from vegetable fats (phytosterols). Examples of zoosterols include cholesterol and lanosterol. Examples of suitable phytosterols include ergosterol, stigmasterol, and sitosterol. There are also sterols that are isolated from fungi and yeasts (so-called mycosterols).
phospholipids. These are understood to mean principally the glucose phospholipids, which are obtained e.g., as lecithins or phosphatidylcholines from, for example, egg yolk or plant seeds (e.g., soybeans),
alkyl (oligo)glycosides. Suitable alkyl (oligo)glycosides can be selected from compounds of the general formula RO-[G]ₓ, in which [G] is preferably derived from aldoses and/or ketoses having 5-6 carbon atoms-preferably from glucose.

The index number x denotes the degree of oligomerization (DP), i.e., the distribution of mono- and oligoglycosides. The index number x preferably has a value in the range from 1 to 10, more preferably in the range of 1 to 3, wherein it need not be a whole number, but can be a fraction which can be determined by analysis.

Particularly preferred alkyl (oligo)glycosides have a degree of oligomerization between 1.2 and 1.5. The functional group R preferably denotes at least one alkyl and/or alkenyl functional group having 4 to 24 C atoms.

Especially preferred alkyl (oligo)glycosides are compounds that are known under the INCl designations Caprylyl/Capryl Glucoside, Decyl Glucoside, Lauryl Glucoside, and Coco Glucoside.

Particularly preferred non-ionic surfactants that can be contained as a co-surfactant in (hair cleaning) agents according to the invention are fatty acid alkanolamides and/or alkyl (oligo) glucosides.

Suitable cationic surfactants in the sense of the present invention are:
quaternary ammonium compounds—for example, selected from compounds of the following formula

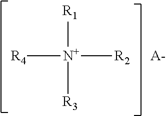

where
the functional groups R¹, R², R³, and R⁴ independently of each other represent hydrogen, a methyl group, a phenyl group, a benzyl group, or a saturated, branched or unbranched alkyl functional group having a chain length of 8 to 30 carbon atoms, which may optionally be substituted with one or more hydroxyl groups, with the proviso that at least one of the functional groups R¹, R², R³, and R⁴ does not represent hydrogen, and
A⁻ represents a physiologically tolerated anion—for example, a halide such as chloride or bromide as well as methosulfates.

Examples of preferred quaternary ammonium compounds of the aforementioned formula are lauryl trimethyl ammonium chloride, cetyl trimethyl ammonium chloride, cetyl trimethyl ammonium bromide, cetyl trimethyl ammonium methosulfate, dicetyl dimethyl ammonium chloride, tricetyl methyl ammonium chloride, stearyl trimethyl ammonium chloride, distearyl dimethyl ammonium chloride, lauryl dimethyl benzyl ammonium chloride, behenyl trimethyl ammonium chloride, behenyl trimethyl ammonium bromide, behenyl trimethyl ammonium methosulfate.

Cetyl trimethyl ammonium chloride and/or behenyl trimethyl ammonium chloride are particularly preferred.
ester quats—for example, selected from compounds of the following formula

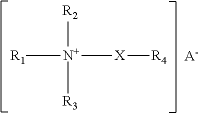

in which the functional groups R¹, R², and R₃ in each case independently of each other can be identical or different and have the following meaning:
a branched or unbranched alkyl functional group having 1 to 4 carbon atoms that can contain at least one hydroxyl group, or
a saturated or unsaturated, branched or unbranched or a cyclically saturated or unsaturated alkyl functional group having 6 to 30 carbon atoms that can contain at least one hydroxyl group, or
an aryl or alkylaryl functional group—for example, phenyl or benzene,
the functional group (—X—R⁴), with the proviso that at most 2 of the functional groups R₁, R₂, and R₃ can represent this functional group, wherein,
X represents:
   1. —(CH₂)ₙ— with n=1 to 20, preferably n=1 to 10, and particularly preferably n=1 to 5, or
   2. —(CH₂—CHR₅—O)ₙ— with n=1 to 200, preferably 1 to 100, particularly preferably 1 to 50, and in particular 1 to 20, and R₅ represents hydrogen, a methyl, or ethyl group,
   3. a hydroxyalkyl group having one to four carbon atoms, which may be branched or unbranched, and which contains at least one and at most 3 hydroxyl groups. Examples are:—CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH, and hydroxybutyl functional groups, and
R₄ represents:
   1. the group R₆—O—CO—, where R₆ represents a saturated or unsaturated, branched or unbranched or a cyclic saturated or unsaturated alkyl functional group having 6 to 30 carbon atoms, which can contain at least one hydroxy group, and which optionally can be oxethylated further with 1 to 100 ethylene oxide units and/or 1 to 100 propylene oxide units, or
   2. the group R₇—O—CO—, where R₇ represents a saturated or unsaturated, branched or unbranched or a cyclic saturated or unsaturated alkyl functional group having 6 to 30 carbon atoms, which can contain at least one hydroxy group, and which optionally can be oxethylated further with 1 to 100 ethylene oxide units and/or 1 to 100 propylene oxide units, and
A represents a physiologically tolerated organic or inorganic anion, e.g., a halide ion such as chloride, bromide, iodide, a sulfate ion of the formula RSO₃_{^{−}} in which R means saturated or unsaturated alkyl functional groups having 1 to 4 carbon atoms, or is an organic acid anion—for example, maleate, fumarate, oxalate, tartrate, citrate, lactate or acetate.
   ester quats—for example, selected from compounds of the following formula

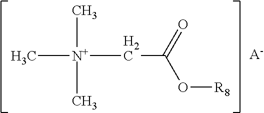

where the meaning of R₈ corresponds to the meaning of R₇ and where A⁻ has the same meaning as described above.

Particular preference is given to ester quats referred to as Distearoylethyl Hydroxyethylmonium Methosulfate, Dicocoylethyl Hydroxyethylmonium Methosulfate, Dipalmitoylethyldimonium Chloride, Behenoyl PG Trimonium Chloride, according to the INCl nomenclature.

In addition to the aforementioned essential and optional constituents, the hair treatment agents according to the invention can contain other hair-conditioning active ingredients in order to enhance their care properties, as long as they do not adversely affect the foaming capacity and/or the stability of the hair treatment agents.

Particularly suitable hair-conditioning active substances within the meaning of the present invention are preferably
d) cationic polymers,
e) oil, fat, and/or wax components,
f) protein hydrolyzates,
g) or mixtures thereof.

In a preferred embodiment, hair treatment agents according to the invention contain at least one cationic polymer d) in a proportion by weight of 0.01-3 wt. %, more preferably 0.02-2 wt. %, and very particularly preferably 0.025-1 wt. % relative to the total weight of the hair treatment agent.

Suitable cationic polymers d) in the sense of the present invention are, for example:
quaternized cellulose polymers—in particular, Polyquaternium-10, such as those commercially available under the names, Celquat® and Polymer JR®,
hydrophobically modified cationic cellulose derivatives—for example, the cationic polymers distributed under the trade name, SoftCat®,
cationic alkylpolyglycosides,
cationized honey—for example, the commercial product, Honeyquat® 50,
cationic galactomannan derivatives—in particular, cationic guar and/or cassia polymers, such as the products available under the trade names, Jaguar®, N-Hance®, Polycare®, Clearhance®. Jaguar® C-162, Jaguar® C₅₀₀, Jaguar® Styl 100, Jaguar® Excel, N-Hance® 3196, N-Hance® HPCG 1000, and/or Polycare® Split Therapy are particularly suitable,
cationic starch derivatives—for example, those commercially available under the name, Mirustyle®,
cationic inulin polymers,
polymeric dimethyldiallyl ammonium salts and copolymers thereof with esters and amides of acrylic acid and methacrylic acid—in particular, Polyquaternium-6 and Polyquaternium-7. The products obtainable commercially under the designations, Merquat®100 (poly(dimethyldiallyl ammonium chloride)) and Merquat®550 (dimethyldiallyl ammonium chloride/acrylamide copolymer) are examples of such cationic polymers,
copolymers of vinylpyrrolidone with quaternized derivatives of dialkylaminoalkyl acrylate and methacrylate, such as with diethylsulfate quaternized vinylpyrrolidone-dimethylaminoethyl methacrylate copolymers. Such compounds are commercially available under the designations, Gafquat®734 and Gafquat®755,
vinylpyrrolidone-vinylimidazolium methochloride copolymers, such as offered under the names, Luviquat® FC 370, FC 550, FC 905, and HM 552,
quaternized polyvinyl alcohol,
as well as the polymers known under the names,
Polyquaternium-2, Polyquaternium-17, Polyquaternium-18, Polyquaternium-24, Polyquaternium-27, Polyquaternium-32, Polyquaternium-37, Polyquaternium-67, Polyquaternium-74, and Polyquaternium-89.

Preferred are cationic polymers of natural origin, such as quaternized cellulose polymers, hydrophobically modified cationic cellulose derivatives and/or cationic guar derivatives; particularly preferred are the cationic polymers known under the INCl names, Guar Hydroxypropyltrimonium Chloride, Polyquaternium-10, Polyquaternium-6, Polyquaternium-37, Polyquaternium-67, or mixtures thereof.

In a further preferred embodiment, hair treatment agents according to the invention additionally contain 0.01-5 wt. %, more preferably 0.025-3 wt. %, and very particularly preferably 0.05-2.5 wt. % of at least one hair care substance e) different from d), wherein the quantities specified relate to the total weight of the hair treatment agent.

Suitable hair care substances e) within the meaning of the present invention are preferably understood to mean oil, fat and/or wax components that can be selected from, for example, glycerol esters, natural oils, butters or waxes, ester oils, ethers, C₈-C₁₆ alkanes, or mixtures thereof.

Suitable oil, fat, and/or wax components e) in the sense of the present invention are, for example:
mineral oils such as paraffin and isoparaffin oils and synthetic hydrocarbons. Examples of hydrocarbons that can be used are, for example, the mixtures of alkanes available as commercial products (Cetiol® Ultimate) and/or 1,3-di-(2-ethylhexyl) cyclohexane (Cetiol® S).
dialkyl ethers, preferably di-n-alkyl ethers having in total between 12 and 36 C atoms, in particular 12 to 24 C atoms, such as for example di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether as well as di-tert-butyl ether, diisopentyl ether, di-3-ethyl decyl ether, tert-butyl n-octyl ether, isopentyl n-octyl ether and 2-methyl pentyl n-octyl ether. Particular preference is given to di-n-octyl ether, which is commercially available under the name Cetiol® OE.
saturated, mono- or polyunsaturated, branched or unbranched fatty alcohols having C₆-C₃₀, preferably C₁₀-C₂₂ and very particularly preferably C₁₂-C₂₂ carbon atoms can be used as fatty alcohols. Examples of this are decanol, octanol, octenol, dodecenol, decenol, octadienol, dodecadienol, decadienol, oleyl alcohol, erucic alcohol, ricinoleic alcohol, stearyl alcohol, isostearyl alcohol, cetyl alcohol, lauryl alcohol, myristyl alcohol, arachidyl alcohol, caprylic alcohol, capric alcohol, linoleyl alcohol, linolenyl alcohol and behenyl alcohol, and Guerbet alcohols thereof, wherein this list is intended to be exemplary and non-limiting in nature. However, the fatty alcohols originate from preferably natural fatty acids, it usually being possible to start from an isolation from the esters of the fatty acids by reduction. According to the invention, use may also be made of those fatty alcohol fractions which are produced by reducing naturally occurring triglycerides such as beef tallow, palm oil, peanut oil, rapeseed oil, cottonseed oil, soybean oil, sunflower oil and linseed oil, or fatty acid esters which are produced from the transesterification products thereof with suitable alcohols, and thus represent a mixture of different fatty alcohols. Such substances are commercially available, for example, under the names Stenol®, e.g. Stenol® 1618 or Lanette®, e.g. Lanette® O or Lorol®, e.g. Lorol® C₈, Lorol® C₁₄, Lorol® C₁₈, Lorol® C₈-18, HD-Ocenol®, Crodacol®, e.g. Crodacol® CS, Novol® Eutanol® G, Guerbitol® 16, Guerbitol@ 18, Guerbitol® 20, Isofol@ 12, Isofol@ 16, Isofol® 24, Isofol® 36, Isocarb® 12, Isocarb® 16 or Isocarb® 24. Of course, lanolin alcohols, such as those commercially available under the names Corona®, White Swan®, Coronet® or Fluilan®, can also be used according to the invention.
ester oils such as the esters of C₆-C₃₀ fatty acids with C₂-C₃₀ fatty alcohols. Preference is given to the monoesters of fatty acids with alcohols having 2 to 24 C atoms. Examples of fatty acid components used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid and erucic acid and technical mixtures thereof. Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol and technical mixtures thereof. Particularly preferred are isopropyl myristate (Rilanit® IPM), isononanoic acid C16-18 alkyl ester (Cetiol® SN), 2-ethylhexyl palmitate (Cegesoft® 24), stearic acid-2-ethylhexyl ester (Cetiol® 868), cetyl oleate, glycerol tricaprylate, coconut fatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).
dicarboxylic acid esters such as di-n-butyl adipate, di-(2-ethylhexyl) adipate, di-(2-ethylhexyl) succinate, and diisotridecyl acelaate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate,
symmetrical, asymmetrical or cyclic esters of carbonic acid with fatty alcohols,
glycerol carbonate or dicaprylyl carbonate (Cetiol® CC),
ethoxylated or non-ethoxylated mono-, di- and trifatty acid esters of saturated and/or unsaturated linear and/or branched fatty acids with glycerol, for example, Monomuls® 90-018 (INCl name: Glyceryl Oleate), Monomuls® 90-L12 (INCl name: Glyceryl Laurate), Cetiol® HE (INCl name: PEG-7 Glyceryl Cocoate) or Cutina® MD (INCl name: Glyceryl Stearate).
natural oils and/or butters, such as coconut oil, (sweet) almond oil, walnut oil, peach kernel oil, apricot kernel oil, avocado oil, tea tree oil, soybean oil, cottonseed oil, sesame oil, sunflower oil, tsubaki oil, evening primrose oil, rice bran oil, palm oil, palm kernel oil, mango kernel oil, cranberry oil, sea buckthorn seed oil, meadow foam oil, thistle oil, macadamia nut oil, grapeseed oil, amaranth seed oil, argan oil, bamboo oil, olive oil, wheat germ oil, pumpkin seed oil, mallow oil, marula oil, pomegranate seed oil, paradise nut oil, passion fruit kernel oil, rose hip kernel oil, hazelnut oil, hemp oil, coffee oil, castor oil, safflower oil, corn oil, olive oil, rapeseed oil, canola oil, sasanqua oil, jojoba oil, rambutan oil, shea butter, mango butter, murumuru butter, cocoa butter, apricot kernel butter, mafura butter, bacuri butter, tucuma butter, ucuuba butter and/or cupuacu butter.
waxes, for example natural waxes such as carnauba wax, candelilla wax and/or jojoba oil, as well as animal waxes such as beeswax, wool wax, spermaceti and/or uropygial gland fat, and/or synthetic mineral waxes such as hard paraffin, ceresin, ozokerite, ester waxes such as polyethylene glycol or polyethylene glycol ester waxes and/or hydrogenated vegetable oils (in particular hydrogenated castor oil).

For some embodiments, it can be particularly advantageous if hair treatment agents according to the invention that are formulated as rinse-off hair cleaning agents contain non-ethoxylated glycerol esters as a care substance e).

In particular, glycerol mono- and/or diesters are suitable in this embodiment because they have a good oil-restoring capacity and nourish the scalp and hair without the occurrence of over-oiling. In addition, they do not impair the foaming properties of such compositions.

A glycerol ester particularly suitable in this context is glycerol monooleate.

In a further preferred embodiment, hair treatment agents according to the invention contain at least one protein hydrolyzate in a proportion by weight of 0.01 to 3 wt. % relative to the total weight of the hair treatment agent.

Suitable protein hydrolyzates are understood to mean product mixtures that can be obtained by acid-, base-, or enzyme-catalyzed degradation of proteins.

Protein hydrolyzates of plant, animal and/or marine origin can be used.

Animal protein hydrolyzates are, for example, elastin, collagen, keratin, silk, and milk protein hydrolyzates, which can also be present in the form of salts. Such products are sold, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Protein hydrolyzates of plant origin are preferred, for example soybean, almond, rice, pea, potato and wheat protein hydrolyzates. Such products are available, for example, under the trade names Gluadin® (Cognis), DiaMin® (Diamalt), Lexein® (Inolex) and Crotein® (Croda).

Cationized protein hydrolyzates can also be used, it being possible for the underlying protein hydrolyzate to originate from animals, e.g., from collagen, milk, or keratin, from plants, e.g., from wheat, corn, rice, potatoes, soybeans, or almonds, from marine life forms, e.g., from fish collagen or algae, or from biotechnologically obtained protein hydrolyzates. The protein hydrolyzates forming the basis of the cationic derivatives may be obtained from the corresponding proteins by chemical, in particular alkaline or acidic, hydrolysis, by enzymatic hydrolysis, and/or by a combination of both types of hydrolysis. The hydrolysis of proteins usually results in a protein hydrolyzate having a molecular weight distribution of approximately 100 daltons up to several thousand daltons. Cationic protein hydrolyzates of which the underlying protein portion has a molecular weight of from 100 to 25,000 daltons, preferably from 250 to 5,000 daltons, are preferred. Furthermore, cationic protein hydrolyzates are understood to mean quaternized amino acids and mixtures thereof. Quaternization of the protein hydrolyzates or of the amino acids is often carried out by means of quaternary ammonium salts such as, for example, N,N-dimethyl-N-(n-alkyl)-N-(2-hydroxy-3-chloro-n-propyl) ammonium halides. Furthermore, the cationic protein hydrolyzates can also be derivatized even further. Mentioned as typical examples of the cationic protein hydrolyzates and derivatives are the products that are commercially available and are known under the INCl names: cocodimonium hydroxypropyl hydrolyzed collagen, cocodimopnium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratain, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, hydroxypropyl arginine lauryl/myristyl ether HCl, hydroxypropyltrimonium gelatin, hydroxypropyltrimonium hydrolyzed casein, hydroxypropyltrimonium hydrolyzed collagen, hydroxypropyltrimonium hydrolyzed conchiolin protein, hydroxypropyltrimonium hydrolyzed keratain, hydroxypropyltrimonium hydrolyzed rice bran protein, hydroxyproypltrimonium hydrolyzed silk, hydroxypropyltrimonium hydrolyzed soy protein, hydroxypropyl hydrolyzed vegetable protein, hydroxypropyltrimonium hydrolyzed wheat protein, hydroxypropyltrimonium hydrolyzed wheat protein/siloxysilicate, laurdimonium hydroxypropyl hydrolyzed soy protein, laurdimonium hydroxypropyl hydrolyzed wheat protein, laurdimonium hydroxypropyl hydrolyzed wheat protein/siloxysilicate, lauryldimonium hydroxypropyl hydrolyzed casein, lauryldimonium hydroxypropyl hydrolyzed collagen, lauryldimonium hydroxypropyl hydrolyzed keratain, lauryldimonium hydroxypropyl hydrolyzed silk, lauryldimonium hydroxypropyl hydrolyzed soy protein, steardimonium hydroxypropyl hydrolyzed casein, steardimonium hydroxypropyl hydrolyzed collagen, steardimonium hydroxypropyl hydrolyzed keratain, steardimonium hydroxypropyl hydrolyzed rice protein, steardimonium hydroxypropyl hydrolyzed silk, steardimonium hydroxypropyl hydrolyzed soy protein, steardimonium hydroxypropyl hydrolyzed vegetable protein, steardimonium hydroxypropyl hydrolyzed wheat protein, steartrimonium hydroxyethyl hydrolyzed collagen, quaternium-76 hydrolyzed collagen, quaternium-79 hydrolyzed collagen, quaternium-79 hydrolyzed keratain, quaternium-79 hydrolyzed milk protein, quaternium-79 hydrolyzed silk, quaternium-79 hydrolyzed soy protein, and quaternium-79 hydrolyzed wheat protein.

Other active substances, auxiliary substances and additives that can preferably be contained in the hair treatment agents according to the invention are, for example:
perfume,
UV filters,
(natural) dyes for coloring the agent,
active ingredients such as bisabolol and/or allantoin,
complexing agents such as EDTA, NTA, β-alanine diacetic acid and phosphonic acids,
ceramides. Ceramides are understood to mean N-acylsphingosine (fatty acid amides of sphingosine) or synthetic analogs of such lipids (so-called pseudo-ceramides),
antioxidants,
preservatives, such as for example sodium benzoate or salicylic acid,
pH setting agents such as citric acid and/or lactic acid,
viscosity regulators such as salts (NaCl).

A second subject matter of the invention is the cosmetic use of an active substance combination comprising
a) at least one compound according to formula (I) and
b) at least one further surfactant different from a) in hair treatment agents for cleaning and caring for human hair, and for the prophylaxis, reduction, elimination, and alleviation of dandruff on hair-covered body surfaces.

Within the second subject matter of the invention, hair treatment agents are preferably to be understood as antidandruff shampoos.

A third subject matter of the invention is a cosmetic method for controlling scalp dandruff in which a hair treatment agent containing
a) at least one compound according to formula (I) and
b) at least one further surfactant different from a) is applied to the hair and the scalp, and
   rinsed out again either after a contact time of 5 seconds to 5 minutes, or
   left on the hair and scalp until the next hair washing.

The statements made about the compositions according to the invention apply in regard to other preferred embodiments of the use according to the invention and the method according to the invention.

The following examples are intended to explain the subject matter of the present invention in a non-limiting manner.

### EXAMPLES

The following hair treatment agents were prepared in which at least one compound according to formula (I) was used in combination with at least one other active antidandruff ingredient b). The quantities specified in the tables are percentages by weight.

| | 1 | 2 | 3 |
|---|---|---|---|
| a) compound according to formula (I) | 0.1-3 | 0.2-2 | 0.3-1.5 |
| b) active antidandruff ingredient different | 0.05-1 | 0.05-0.75 | 0.05-0.5 |
| from a) | | | |
| Water and, optionally, further auxiliaries | up to | up to | up to |
| and active ingredients | 100 | 100 | 100 |

| | 4 | 5 | 6 |
|---|---|---|---|
| a) compound according to formula (I) | 0.1-3 | 0.2-2 | 0.3-1.5 |
| b) active antidandruff ingredient different | 0.05-1 | 0.05-0.75 | 0.05-0.5 |
| from a) | | | |
| Water and optionally further auxiliaries | up to | up to | up to |
| and active ingredients | 100 | 100 | 100 |

| | 7 | 8 | 9 |
|---|---|---|---|
| a) methylheptylglycerin | 0.1-3 | 0.2-2 | 0.3-1.5 |
| b) active antidandruff ingredient different | 0.05-1 | 0.05-0.75 | 0.05-0.5 |
| from a) | | | |
| Water and optionally further auxiliaries | up to | up to | up to |
| and active ingredients | 100 | 100 | 100 |

| | 10 | 11 | 12 |
|---|---|---|---|
| a) methylheptylglycerin | 0.1-3 | 0.2-2 | 0.3-1.5 |
| b) piroctone olamine, zinc pyrithione, | 0.05-1 | 0.05-0.75 | 0.05-0.5 |
| zinc oxide, zinc carbonate, climbazole, | | | |
| ketoconazole, salicylic acid, sulfur, selenium | | | |
| sulfide, tar preparations, undecenoic acid | | | |
| derivatives, lactic acid esters (Sodium Lauroyl | | | |
| Lactylate, Sodium Caproyl Lactylate, Sodium | | | |
| Caproyl/Lauroyl Lactylate), 3- | | | |
| hydroxypropyloctanoates (Propanediol | | | |
| Caprylate), burdock root extracts, poplar | | | |
| extracts, stinging nettle extracts, walnut shell | | | |
| extracts, birch extracts, willow bark extracts, | | | |
| beard lichen extracts, myrtle extracts, | | | |
| hazelwort extracts, rosemary extracts, arnica | | | |
| extracts, fruit extracts of black pepper (Piper | | | |
| Nigrum Fruit Extract), bark extracts of mimosa | | | |
| plants (Inga Alba Bark Extract), extracts from | | | |
| cinnamon bark (Cinnamomum Cassia Bark | | | |
| Extract), tangerine peel extracts (Citrus | | | |
| Reticulata (Tangerine) Peel Extract), lemon | | | |
| flower oil, lemon leaf oil, lemon fruit oils (Citrus | | | |
| Limon (Lemon) Flower/Leaf/Stem Oil), extracts | | | |
| from mint (Mentha Arvensis Extract), or | | | |
| mixtures thereof. | | | |
| Water and optionally further auxiliaries and | up to | up to | up to |
| active ingredients | 100 | 100 | 100 |

The active ingredients a) and b) were used in a weight ratio of 5:1 to 1:1.

| | 13 | 14 | 15 |
|---|---|---|---|
| a) methylheptylglycerin | 0.1-3 | 0.2-2 | 0.3-1.5 |
| b) piroctone olamine | 0.05-1 | 0.05-0.75 | 0.05-0.5 |
| Water and optionally further auxiliaries | up to | up to | up to |
| and active ingredients | 100 | 100 | 100 |

| | 16 | 17 | 18 |
|---|---|---|---|
| a) methylheptylglycerin | 0.1-3 | 0.2-2 | 0.3-1.5 |
| b) propanediol caprylate | 0.05-1 | 0.05-0.75 | 0.05-0.5 |
| Water and optionally further auxiliaries | up to | up to | up to |
| and active ingredients | 100 | 100 | 100 |

| | 19 | 20 | 21 |
|---|---|---|---|
| a) methylheptylglycerin | 0.1-3 | 0.2-2 | 0.3-1.5 |
| b) sodium caproyl/lauroyl lactylate | 0.05-1 | 0.05-0.75 | 0.05-0.5 |
| Water and optionally further auxiliaries | up to | up to | up to |
| and active ingredients | 100 | 100 | 100 |

| | 22 | 23 | 24 |
|---|---|---|---|
| a) compound according to formula (I) | 0.1-3 | | |
| Methylheptylglycerin | | 0.2-2 | 0.3-1.5 |
| b) active antidandruff ingredient different | 0.05-1 | | |
| from a) | | | |
| piroctone olamine, zinc pyrithione, zinc oxide, | | 0.05-0.75 | 0.05-0.5 |
| zinc carbonate, climbazole, ketoconazole, | | | |
| salicylic acid, sulfur, selenium sulfide, tar | | | |
| preparations, undecenoic acid derivatives, | | | |
| lactic acid esters (Sodium Lauroyl Lactylate, | | | |
| Sodium Caproyl Lactylate, Sodium | | | |
| Caproyl/Lauroyl Lactylate), 3- | | | |
| hydroxypropyloctanoates (Propanediol | | | |
| Caprylate), burdock root extracts, poplar | | | |
| extracts, stinging nettle extracts, walnut shell | | | |
| extracts, birch extracts, willow bark extracts, | | | |
| beard lichen extracts, myrtle extracts, | | | |
| hazelwort extracts, rosemary extracts, arnica | | | |
| extracts, fruit extracts of black pepper (Piper | | | |
| Nigrum Fruit Extract), bark extracts of mimosa | | | |
| plants (Inga Alba Bark Extract), extracts from | | | |
| cinnamon bark (Cinnamomum Cassia Bark | | | |
| Extract), tangerine peel extracts (Citrus | | | |
| Reticulata (Tangerine) Peel Extract), lemon | | | |
| flower oil, lemon leaf oil, lemon fruit oils (Citrus | | | |
| Limon (Lemon) Flower/Leaf/Stem Oil), extracts | | | |
| from mint (Mentha Arvensis Extract), or | | | |
| mixtures thereof. | | | |
| Surfactant | 0.5-20 | | |
| Anionic surfactant | | 1-15 | 2.5-15 |
| Amphoteric or zwitterionic surfactant | | 0-7.5 | 0.1-5 |
| Non-ionic surfactant | | 0-5 | 0-3 |
| Water and optionally further auxiliaries and | up to | up to | up to |
| active ingredients | 100 | 100 | 100 |

| | 25 | 26 | 27 |
|---|---|---|---|
| Methylheptylglycerin | 0.1-3 | 0.1-3 | 0.1-3 |
| Piroctone olamine | 0.05-1 | | |
| Propanediol caprylate | | 0.05-1 | |
| Sodium caproyl/lauroyl lactylate | | | 0.05-1 |
| Anionic surfactant | 1-15 | 1-15 | 1-15 |
| Amphoteric or zwitterionic surfactant | 0-7.5 | 0-7.5 | 0-7.5 |
| Non-ionic surfactant | 0-5 | 0-5 | 0-5 |
| Cationic polymer d) | 0-3.5 | 0-3.5 | 0-3.5 |
| Care substance e) | 0-5 | 0-5 | 0-5 |
| Water and optionally further | up to | up to | up to |
| auxiliaries and active ingredients | 100 | 100 | 100 |

The active ingredients a) and b) were used in a weight ratio of 5:1 to 1:1.

| | 28 | 29 | 30 | 31 |
|---|---|---|---|---|
| Methylheptylglycerin | 0.1-3 | 0.2-2 | 0.3-1.5 | 0.5 |
| Piroctone olamine | 0.05-1 | | | 0.2 |
| Propanediol caprylate | | 0.05-1 | | |
| Sodium caproyl/lauroyl | | | 0.05-1 | |
| lactylate | | | | |
| Sodium laureth sulfate | 1-15 | 1-15 | 1-15 | 9 |
| Cocamidopropyl betaine | 0-7.5 | 0-7.5 | 0-7.5 | 1.5 |
| Lamesoft ® PO 65* | 0-5 | 0-5 | 0-5 | 1.2 |
| Guar | 0-3.5 | 0-3.5 | 0-3.5 | 0.05 |
| hydroxypropyltrimonium | | | | |
| chloride | | | | |
| Water and optionally | up to | up to | up to | |
| further auxiliaries and | 100 | 100 | 100 | |
| active ingredients | | | | |

The active ingredients a) and b) were used in a weight ratio of 5:1 to 1:1.
INCl name: Coco Glucoside and Glyceryl Oleate (approx. 65% AS); BASF

The composition 31 according to the invention has the following advantages over compositions which, instead of 0.5 wt. % of methylheptylglycerin +0.2 wt. % of piroctone olamine contain only 0.3 wt. % of piroctone olamine:
it has an at least equivalent antidandruff action,
it has a lower potential for skin irritation,
it has improved environmental compatibility,
it has at least equivalent care properties.

What is claimed is:

1. A hair treatment agent, including a cosmetic carrier, comprising
a) at least one compound of formula (I)
R¹—O—[CH₂—CH(OH)—CH₂—O]ₙ—H (I)
where
R¹ represents a branched C₆-C₁₆ alkyl, alkenyl, or alkynyl group
n represents a number from 1-3, and
b) at least one active anti-dandruff ingredient different from the at least one compound of formula I a).

2. The hair treatment agent according to claim 1, wherein the at least one compound of formula I a) and b) are present in the agent in a weight ratio ranging from 5:1 to 1:1.

3. The hair treatment agent according to claim 1, wherein the total amount of the at least one compound of formula (I) is 0.1-3 wt. %, relative to the total weight of the hair treatment agent.

4. The hair treatment agent according to claim 1, wherein n is the number 1.

5. The hair treatment agent according to claim 1, wherein, in R¹ represents a branched C₆-C₁₂ alkyl, alkenyl, or alkynyl group.

6. The hair treatment agent according to claim 1, wherein the at least one compound of formula (I) is H₃C—(CH₂)₅—CH(CH₃)—O—CH₂—CH(OH)—CH₂OH (methylheptylglycerin).

7. The hair treatment agent according to claim 1, wherein the total amount of the at least one active anti-dandruff ingredient b) different from a) is 0.05-1 wt. %, relative to the total weight of the hair treatment agent.

8. The hair treatment agent according to claim 1, wherein the at least one active anti-dandruff ingredient b) different from the at least one compound of formula I is selected from the group consisting of: piroctone olamine, zinc pyrithione, zinc oxide, zinc carbonate, climbazole, ketoconazole, salicylic acid, sulfur, selenium sulfide, tar preparations, undecenoic acid derivatives, lactic acid esters (Sodium Lauroyl Lactylate, Sodium Caproyl Lactylate, Sodium Caproyl/Lauroyl Lactylate), 3-hydroxypropyloctanoates (Propanediol Caprylate), burdock root extracts, poplar extracts, stinging nettle extracts, walnut shell extracts, birch extracts, willow bark extracts, beard lichen extracts, myrtle extracts, hazelwort extracts, rosemary extracts, arnica extracts, fruit extracts of black pepper (Piper nigrum Fruit Extract), bark extracts of mimosa plants (Inga Alba Bark Extract), extracts from cinnamon bark (Cinnamomum cassia Bark Extract), tangerine peel extracts (Citrus reticulata (Tangerine) Peel Extract), lemon flower oil, lemon leaf oil, lemon fruit oil (Citrus limon (Lemon) Flower/Leaf/Stem Oil), extracts from mint (Mentha arvensis Extract), and mixtures thereof.

9. The hair treatment agent according to claim 1, wherein the at least one active anti-dandruff ingredient different from the at least one compound of formula I is selected from the group consisting of piroctone olamine, propanediol caprylate, sodium caproyl/lauroyl lactylate, and mixtures thereof.

10. The hair treatment agent according to claim 1, further comprising 0.5-20 wt. % of at least one surfactant c) selected the group consisting of: from anionic surfactants, amphoteric surfactants, zwitterionic surfactants, non-ionic surfactants, cationic surfactants, and mixtures thereof.

11. The hair treatment agent according to claim 1, further comprising 0.01-3 wt. % of at least one cationic polymer d), wherein the quantities specified relate to the total weight of the hair treatment agent.

12. The hair treatment agent according to claim 1, further comprising 0.01-5 wt. % of at least one hair care substance e) different from d), wherein the quantities specified relate to the total weight of the hair treatment agent.

13. The hair treatment agent according to claim 1, wherein the hair treatment agent is an anti-dandruff shampoo.

14. A cosmetic method for controlling scalp dandruff, comprising applying the hair treatment agent of claim 1 to hair or scalp, and optionally, rinsing the hair or scalpe out again after a contact time of 5 seconds to 5 minutes.
